(19) United States
(12) Patent Application Publication
(10) Pub. No.: US 2023/0098927 A1
(43) Pub. Date: Mar. 30, 2023

(54) COMPOSITION SUITABLE FOR USE IN A DIAGNOSTIC METHOD TO DIAGNOSE PLAQUE FORMATION AND COMPUTER-IMPLEMENTED DIAGNOSTIC METHOD

(71) Applicants: SOCIETE DES PRODUITS NESTLE S.A., Vevey (CH); SINGAPORE INSTITUTE FOR CLINICAL SCIENCES, BIOMEDICAL SCIENCES INSTITUTES, Singapore (SG); SINGAPORE HEALTH SERVICES PTE LTD, Singapore (SG)

(72) Inventors: LUCAS ACTIS GORETTA, Dublin, OH (US); ARMAND VALSESIA, Dublin, OH (US); LEONIE EGLI, La Tour-de-Peilz (CH); FABRIZIO ARIGONI, Lausanne (CH); DIANE ZIMMERMANN, Feigeres (FR); NASHMIL EMAMI, Raymond, OH (US); MOHAMED NABIL BOSCO, Delaware, OH (US); KHEE SHING MELVIN LEOW, Singapore (SG); KHUNG KEONG YEO, Singapore (SG)

(21) Appl. No.: 17/907,010

(22) PCT Filed: Mar. 29, 2021
(86) PCT No.: PCT/EP2021/058115

§ 371 Date: Sep. 22, 2022

(30) Foreign Application Priority Data
Mar. 27, 2020 (EP) 20166317.6

(51) Int. Cl. A23L 33/115; A23L 33/19

(52) U.S. Cl. CPC A23L 33/115; A23L 33/19

(57) ABSTRACT

The present invention relates generally to the field of compositions for use in diagnostic methods. In particular, the present invention relates to a composition for use in a diagnostic method wherein the composition is a nutritional composition comprising 15-70 g fat, 60-90 g carbohydrates and 15-35 g protein. The diagnostic method may be a method to predict the risk of atheroma plaque presence in a subject.

The present invention relates generally to the field of compositions for use in diagnostic methods. In particular, the present invention relates to a composition for use in a diagnostic method wherein the composition is a nutritional composition comprising 15-70 g fat, 60-90 g carbohydrates and 15-35 g protein. The diagnostic method may be a method to predict the risk of atheroma plaque formation in a subject.

Cardiovascular diseases (CVD) remain the leading cause of mortality today, and the Global Burden of Disease Study reported almost 18 million deaths from CVD worldwide in 2015. Environmental factors related to lifestyle, such as unhealthy dietary habits and lack of regular physical activity, undeniably play an important role in the development of CVD and thus make it largely preventable. Indeed, several epidemiological studies showed that adoption of a healthy diet pattern, such as the Mediterranean diet, for example, helps to reduce the incidence of cardiovascular events.

Classical risk indicators, such as fasting cholesterol levels, blood pressure and diabetes status are used today to predict the risk of developing CVD. These risk indicators have been successfully used to develop several risk scores to assess CVD or coronary heart disease (CHD) risks. However, the ability to accurately predict these disease risks remains limited, particularly in lower risk groups such as women and younger populations.

Recently, it was shown (J Am Coll Cardiol. 2017 Dec 19; 70(24):2979-2991) that among the apparently healthy, middle-aged participants of the PESA study (Progression of Early Subclinical Atherosclerosis, Spain), subclinical atherosclerosis (defined here as presence of plaque in the carotid, abdominal aortic or iliofemoral territory or a coronary artery calcification score 1) was present in 63% of the participants. Interestingly, 58% of the participants who were classified as having low 10-year risk of coronary heart disease from the Framingham Heart Study (FHS) had already developed subclinical atherosclerosis in at least one of the vascular sites assessed. These results demonstrate that subclinical atherosclerosis is already prevalent in an apparently healthy and relatively young population, even among low-risk individuals.

Additional risk indicators should be added to the existing risk scores to better predict the risk of CVD. Indeed, it was further shown (J Am Coll Cardiol. 2015 Mar 24;65(11):1065-74) that quantification of subclinical carotid and coronary atherosclerosis improves CVD risk prediction. Moreover, subclinical atherosclerosis measured by carotid Intima-Media Thickness (cIMT) and cardiac structure and/or function are also known to be linked to cardiovascular risk and death and might improve CVD risk prediction.

While elaborate tests to assess subclinical atherosclerosis or cardiac structure and function could theoretically be added to the traditional CVD risk scales, this would be a time-consuming and costly approach.

Although people are aware that certain lifestyle changes, such as increasing physical activity and adapting to a healthier nutrition has a positive impact on preventing cardiovascular diseases, the prevalence of cardiovascular diseases continues to increase.

One main reason appears to be that even though the general knowledge about healthy nutrition and lifestyle has increased in the past decades, consumers still lack a tangible way to see an immediate effect on their cardiovascular health. Given the lengthy process of disease development and progression, it would be desirable to have available a feedback tool based on acute physiological responses to guide and motivate consumers.

Further, it would be desirable to have available a tool that allows the prediction of the development of cardiovascular diseases in a cost effective and fast way.

Any reference to prior art documents in this specification is not to be considered an admission that such prior art is widely known or forms part of the common general knowledge in the field.

The objective of the present invention was it to enrich or improve the state of the art, and, in particular, to provide a composition that can be used in a diagnostic method that allows it after consumption of the composition to predict the likelihood to develop cardiovascular diseases and/or the risk to develop atheroma plaque formation and/or the risk to have already formed atheroma plaques in a subject, or to at least provide a useful alternative to solutions available in the prior art.

The inventors were surprised to see that the objective of the present invention could be achieved by the findings presented in this document and, in particular, by the subject matter of the independent claims. The dependent claims further develop the idea of the present invention.

Accordingly, the present invention provides a diagnostic method to predict the risk of developing cardiovascular diseases and/or the risk to have already formed atheroma plaques in a subject.

The present invention further provides a composition for use in a diagnostic method, for example a diagnostic method to predict the risk of developing cardiovascular diseases and/or the risk to have already formed atheroma plaques in a subject.

As used in this specification, the words “comprises”, “comprising”, and similar words, are not to be interpreted in an exclusive or exhaustive sense. In other words, they are intended to mean “including, but not limited to”.

The inventors have shown that a predictive tool can be created that is based on postprandial biomarkers, such as blood pressure after consumption of a specific nutritional composition, and that allows to predict atheroma plaque presence, for example, as a proxy surrogate endpoint of cardiovascular diseases.

The nutritional composition should comprise proteins, fat and carbohydrates in sufficient amounts to be able to capture all metabolic signals in relation to these macronutrients.

In particular, the inventors could demonstrate that the consumption of a nutritional composition comprising proteins, fat and carbohydrates allowed it to determine the likelihood of atheroma plaque presence in a subject based on the blood pressure measured after consumption of the nutritional composition. The determination of likelihood was found to be more precise, if the age of the subject was taken into account.

Consequently, the consumption of the composition of the present invention followed by blood pressure determination and under consideration of the age of the subject allows it to predict the likelihood to develop cardiovascular disorders.

Advantageously, the administration of the composition of the present invention followed by blood pressure determination and under consideration of the age of the subject allows it to predict the likelihood to develop cardiovascular disorders in a fast and cost-efficient way, which is non-invasive. The fact that the present invention allows it to make such a prediction fast, non-invasively and inexpensively allows it further that subjects can monitor the positive effect that lifestyle changes and changes to food consumption have on the likelihood to develop cardiovascular disorders. Such a fast and efficient way to monitor progress will be rewarding and motivating to these subjects. Further, as such a prediction can be carried out inexpensively, the benefits of such a prediction can be offered to a much broader range of subjects.

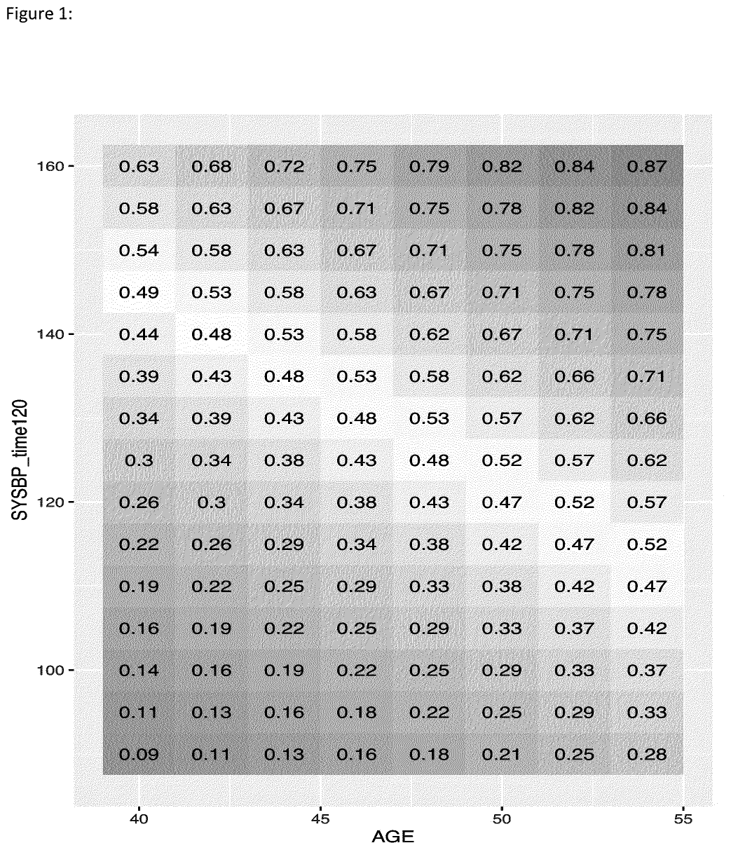

FIG. 1 shows a decision matrix for systolic blood pressures obtained after consumption of the composition of the present invention. The linear equation for the straight line −0.002×the mean-centered and unit-scaled age of the subject +0.57 is indicated by the white boxes, with every value above the white line indicating an increased risk for atheroma plaque presence.

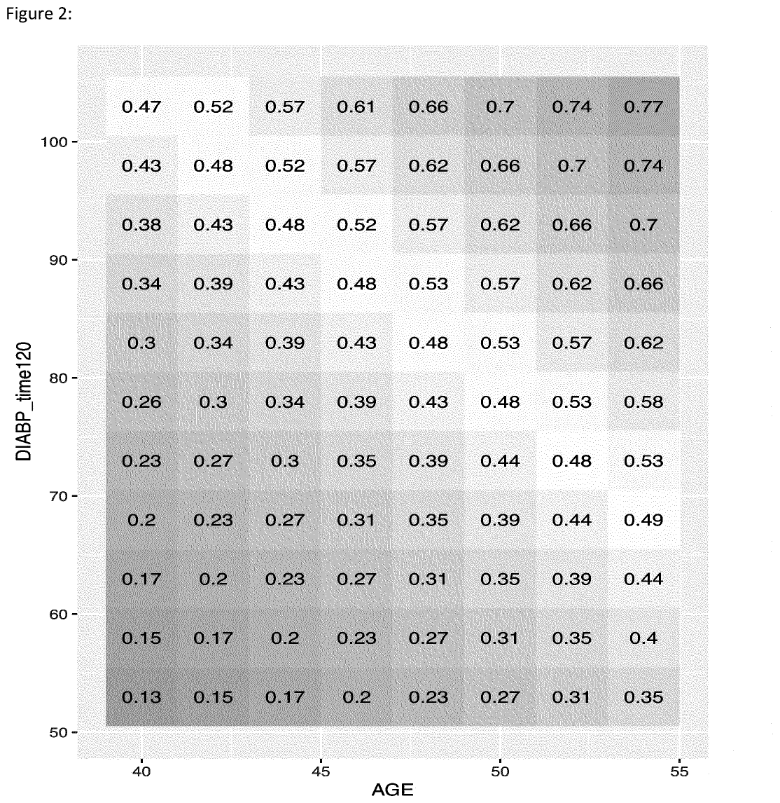

FIG. 2 shows a decision matrix for diastolic blood pressures obtained after consumption of the composition of the present invention. The linear equation for the straight line 0.001×the mean-centered and unit-scaled age of the subject +0.43 is indicated by the white boxes, with every value above the white line indicating an increased risk for atheroma plaque presence.

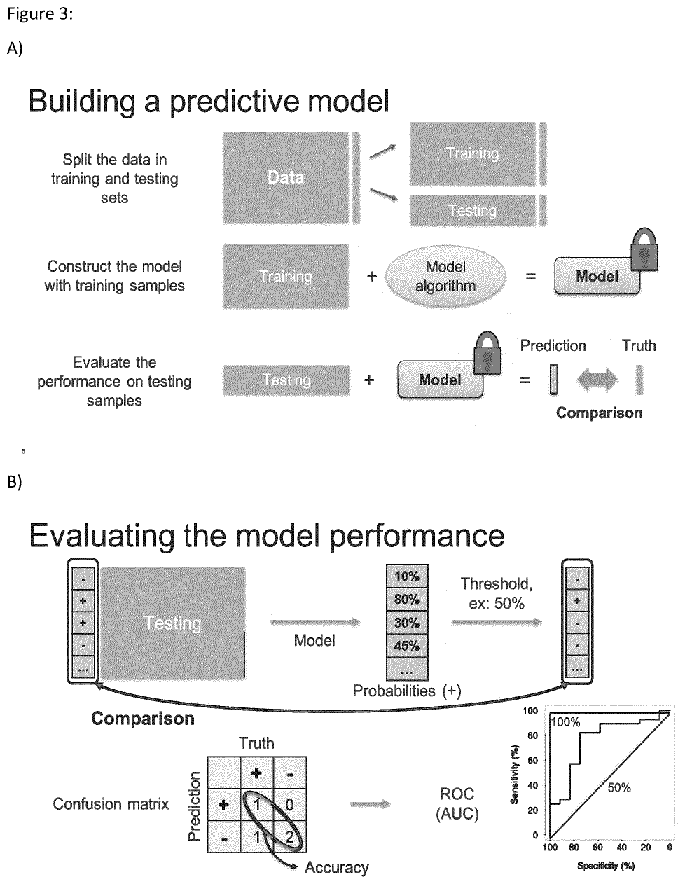

FIG. 3A outlines the construction and training of a classification model. FIG. 3B shows the principles of performance evaluation for the training phase and final validation of trained models.

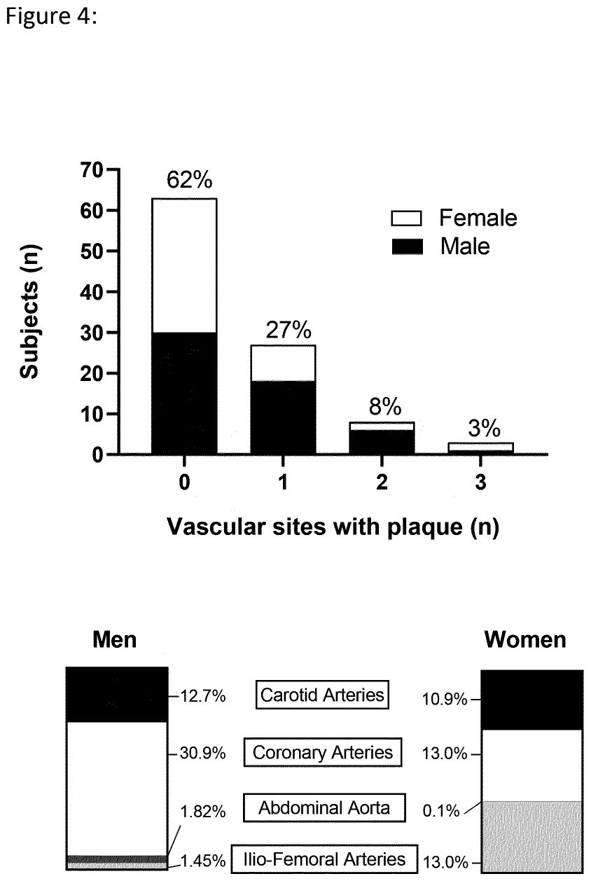

FIG. 4 shows the results of the subclinical atherosclerosis and cardiovascular assessments.

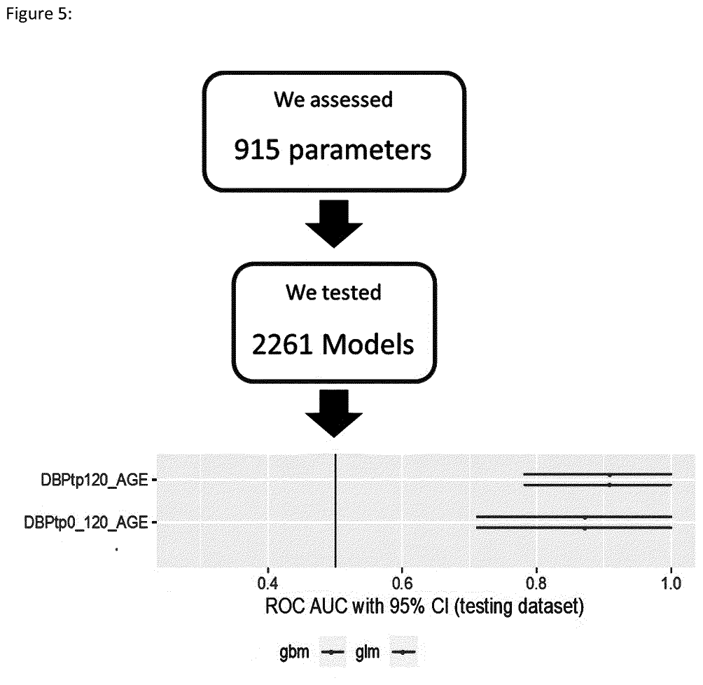

FIG. 5 shows that the food challenge allows it to select blood pressure as a powerful biomarker to predict the presence or absence of atheroma plaque with 91% confidence.

Consequently the present invention relates in part to a composition for use in a diagnostic method, wherein the composition is a nutritional composition comprising 15-70 g fat, 60-90 g carbohydrates and 15-35 g protein.

The composition may be any type of composition suitable for consumption for the subject to whom it is to be administered. The subject may be a mammal, in particular a human, for example. The human may be a male and/or a female human. For example, the human may be an adult, for example, an adult that is 18 years old or older. Further, for example, the adult may be 30 years old or older, 40 years old or older, or 50 years old or older. According to an embodiment of the present invention, the adult may be from 18-99 years old, preferably from 40-54 years old. The clinical study carried out by the inventors focused on adults with an age in the range of 40-54 years.

For the purpose of the present invention, the term “nutritional composition”, as used herein, means any composition that can be used to provide nutrition to a subject. Typically, nutritional compositions contain a protein source, a carbohydrate source and a lipid source.

Said nutritional composition may further comprise any other ingredient, for example, one or more ingredients set out herein e.g. probiotics, vitamins and/or minerals. The composition may also comprise other ingredients commonly used in the form of composition in which it is employed, e.g., a powdered nutritional supplement, a food product, a dairy product, or a drink. Non-limiting examples of such ingredients include: other nutrients, for instance, selected from the group consisting of lipids (optionally in addition to DHA and ARA), carbohydrates, proteins, micronutrients (in addition to those set out above), and/or pharmaceutically active agents; conventional food additives such as anti-oxidants, stabilizers, emulsifiers, acidulants, thickeners, buffers or agents for pH adjustment, chelating agents, colorants, excipients, flavor agents, osmotic agents, pharmaceutically acceptable carriers, preservatives, sugars, sweeteners, texturizers, emulsifiers, water and any combination thereof.

It is preferred that the nutritional composition is dense in macronutrients, as more pronounced effects will be obtained, if sufficient amounts of macronutrients are consumed. Further, the composition for use in accordance with the present invention may be calorically dense. For example, the nutritional composition may contain 500—1000 kcal, for example 550-950 kcal per serving.

The composition for use in accordance with the present invention may comprise 20-71 g fat. The composition for use in accordance with the present invention may comprise 70-80 g carbohydrates. The composition for use in accordance with the present invention may comprise 20-30 g protein. For example, the nutritional composition comprises 20-71 g fat, 70-80 g carbohydrates and 20-30 g protein.

The composition for use in accordance with the present invention may have a relatively high fat content. A high fat content has the advantage that the measured effect will be more pronounced. Hence, in one embodiment, the nutritional composition may comprise 55-71 g fat, 70-80 g carbohydrates and 20-30 g protein.

However, consumers often dislike compositions with a very high fat content. Hence, to ensure consistent usage, it may be preferred to use compositions with a lower, but still sufficiently high, fat content. Hence, in a further embodiment, the nutritional composition may comprise 20-30 g fat, 70-80 g carbohydrates and 20-30 g protein.

The composition of the present invention may further comprise vitamins and minerals. The vitamins and minerals may be selected from the group consisting of vitamin A, vitamin D, vitamin E, vitamin K, vitamin C, thiamin, riboflavin, niacin, vitamin B6, folic acid, vitamin B12, pantothenic acid, potassium, calcium, phosphorus, magnesium, iron, zinc, selenium, or combinations thereof.

The composition for use in accordance with the present invention may be a composition that can be easily consumed by the subject to which the composition is to be administered. For example, the composition may have the form of a bar or a spoonable composition. However, consumer research has shown that it might be preferred, if the composition to be used in accordance with the present invention is a drink.

It terms of volume, the composition of the present invention should have a quantity that can be consumed relatively easily in one consumption occasion. Hence, the volume of the composition can be adjusted accordingly. A person skilled in the art will be able to do so.

For example, the composition may be a drinkable composition with a volume in the range of 200 ml-400 ml. For hygienic purposes it may be preferred if the composition of the present invention is provided in a single serve container.

The inventors have found that the composition of the present invention can effectively be used in a diagnostic method to determine the risk of developing a cardiovascular disease.

In particular, the composition to be used in the framework of the present invention was effective in triggering a metabolic response, which could be assessed by measuring the blood pressure of the subject after the nutritional challenge. The determined blood pressure, optionally in combination with the age of the tested subject allowed it to determine the risk of developing a cardiovascular disease, in particular the risk of developing or having developed atheroma plaque. The risk of atheroma plaque formation may be used as an indicator to predict the likelihood of the presence or development of cardiovascular disorders.

Hence, the diagnostic method may be a diagnostic method to predict the risk of developing cardiovascular disorders in a subject. For example, the diagnostic method may be a diagnostic method to predict the risk of developing atherosclerosis in a subject. Further, for example, the diagnostic method may be a diagnostic method to predict the risk of atheroma plaque formation in a subject. The diagnostic method may also be a diagnostic method to predict the risk of subclinical atherosclerosis in a subject.

The inventors have shown that after administration of the composition of the present invention it is possible to accurately predict the risk of having developed atheroma plaques in a subject.

Atheroma plaques are accumulations of material in the inner layer of the wall of an artery. Such material may include lipids, calcium, macrophage cells, debris, and fibrous connective tissue, for example. Atheroma plaques may result in a narrowing of the channel of the artery, which in turn may result in a restriction of blood flow. Consequently, atheroma plaques are a basis for cardiovascular disorders, in particular arteriosclerosis, for example atherosclerosis.

The diagnostic method may comprise the steps of administering to the subject the composition of the present invention, and measuring the blood pressure of that subject after administration of the composition, wherein an elevated blood pressure compared to a reference value indicates an increased risk for atheroma plaque formation.

The blood pressure that is measured in the framework of the diagnostic method may be the systolic blood pressure (SYSBP) and/or the diastolic blood pressure (DIABP). If the blood pressure measured is the systolic blood pressure, the reference value should also relate to the systolic blood pressure. If the blood pressure measured is the diastolic blood pressure, the reference value should also relate to the diastolic blood pressure.

The present inventors have obtained particular good results, if the age of the subject was also taken into account. Hence, the diagnostic method can include the determination of the age of the subject and the measured blood pressure is compared to a reference value specific for the age group of the subject.

The inventors believe that the task of comparing the measured blood pressure to a reference value while taking the age of the subject into account is most efficiently done by using an algorithm that allows it to enter measured blood pressure and age and produces based thereon a risk factor, for example a risk factor expressed in %. Hence, the diagnostic method may further include that the measured blood pressure and the age of the subject are subjected to an algorithm that produces an indicative figure and that indicative figure is compared to a corresponding indicative reference figure for the age group of the subject.

To achieve optimal comparability of the measured data, the diagnostic method may further include that the measured blood pressure (DIABP and/or SYSBP, respectively) and the age of the subject are mean-centered and unit-scaled.

In general, a value can be mean-centered and unit-scaled by subtracting the mean value from the measured value and dividing the result by the standard deviation.

Concretely, this may mean for the

Age: agenew=(age −47.9)/4.2

Diastolic blood pressure: DIABPnew=(DIABP −71.4)/10.0

Systolic blood pressure: SYSBPnew=(SYSBP −114.4)/13.5

, wherein “new” indicates the mean-centered and unit-scaled value.

A person skilled in the art will be able to compute a logit score from these mean-centered and unit-scaled values using the coefficients below.

| | DIABP | SYSBP |
|---|---|---|
| Intercept | 0.5108 | 0.5253 |
| DIABP | −0.3682 | — |
| SYSBP | — | −0.5403 |
| Age | −0.3998 | −0.3996 |

The logit score can then be converted into a probability using the standard method for logistic regression.

The diagnostic method may further include that the postprandial systolic blood pressure of the subject is measured and an increased risk of atheroma plaque formation is predicted, if the value for the mean-centered and unit-scaled systolic blood pressure is bigger than −0.002×the mean-centered and unit-scaled age of the subject +0.57.

The diagnostic method or at least one step of the diagnostic method may further be implemented on a computer machine or through any digital system. In an embodiment, a data processing device may comprise means for carrying out the computer/digital implemented method described herein. In another embodiment, a computer readable medium may comprise instructions which, when executed by a data processing device, such as a computer, cause the data processing device to carry out the methods described herein.

The system includes a user device and a recommendation system. The user device may be implemented as a computing device, such as a computer, smartphone, tablet, smartwatch, or other wearable through which an associated user can communicate with the recommendation system. The recommendation system includes one or more of a display, an attribute receiving unit, an attribute comparison unit, an attribute analysis unit, an attribute storing unit, a memory, and a CPU. Note, that in some embodiments, a display may additionally or alternatively be located within the user device.

In an embodiment, the device is a client device. The client device is any device that can access content provided or served by a host device. For example, the client device may be any device that can run a suitable web browser to access a web-based interface to the host device. Alternatively or in addition, one or more applications or portions of applications that provide some of the functionality described herein may operate on the client device, in which case the client device is required to interface with the host device merely to access data stored in the host device. In one embodiment, host device is a device that provides cloud-based services, such as cloud-based authentication and access control, storage, streaming, and feedback provision.

FIG. 1 shows a corresponding decision matrix. Accordingly, the diagnostic method may further include that an increased risk of atheroma plaque formation is predicted if (systolic blood pressure−114.4)/13.5 >−0.002×(age of the subject−47.9)/4.2)+0.57. The linear equation for the straight line −0.002×the mean-centered and unit-scaled age of the subject +0.57 is indicated by the white boxes, with every value above the white line indicating an increased risk for atheroma plaque formation.

The diagnostic method may further include that the postprandial diastolic blood pressure of the subject is measured and an increased risk of atheroma plaque formation is predicted, if the value for the mean-centered and unit-scaled diastolic blood pressure is bigger than 0.001× the mean-centered and unit-scaled age of the subject +0.43.

FIG. 2 shows a corresponding decision matrix. Accordingly, the diagnostic method may further include that an increased risk of atheroma plaque formation is predicted if (diastolic blood pressure −71.4)/10 >0.001× (age of the subject −47.9)/4.2)+0.43. The linear equation for the straight 0.001× the mean-centered and unit-scaled age of the subject +0.43 is indicated by the white boxes, with every value above the white line indicating an increased risk for atheroma plaque formation.

The inventors have found that the effects of the composition of the present invention are most pronounced, if sufficient time is allowed between the administration of the composition of the present invention and the measurement of the blood pressure. A person skilled in the art will be able to determine such a time period that allows the composition of the present invention to have optimum effects. The present inventors have found particular good results, if the blood pressure was measured 65−360 min, for example 90−150 minutes, further for example 110−130 minutes after the nutritional composition was administered. In one embodiment, the blood pressure was measured 120 min after the nutritional composition was administered.

Those skilled in the art will understand that they can freely combine all features of the present invention disclosed herein. In particular, features described for the composition of the present invention may be combined with features described for the diagnostic method described in the framework of the present invention and vice versa. Further, features described for different embodiments of the present invention may be combined.

Although the invention has been described by way of example, it should be appreciated that variations and modifications may be made without departing from the scope of the invention as defined in the claims.

Furthermore, where known equivalents exist to specific features, such equivalents are incorporated as if specifically referred in this specification. Further advantages and features of the present invention are apparent from the figures and non-limiting examples.

### EXAMPLES

Methods

Subjects: One hundred and one healthy Chinese subjects (46 women, 55 men) participated and completed this study. Inclusion criteria were: a) willing and able to sign written informed consent in English or Chinese prior to trial entry; b) 40−54 years old; c) both male and female subjects; d) Chinese ethnic group (having both grandparents Chinese); e) Low Framingham risk of CHD (<10%); f) apparently healthy, based on investigator's clinical judgement.

Exclusion criteria were: a) food allergy to any of the constituents of the meal challenge (milk proteins, lactose, soy); b) subjects not willing or not able to comply with scheduled visits and the requirements of the study protocol; c) contraindication to MRI (i.e. cardiac pacemaker, brain aneurysm or clips, electronic implants or prosthesis and others); e) pregnant or lactating women, based on investigator's clinical judgement; f) morbid obesity (BMI 40 kg/m2); g) previous myocardial infarction (MI); h) known coronary artery disease - prior coronary revascularization; i) known documented peripheral arterial disease; j) previous stroke (defined as new focal neurological deficit persisting more than 24 hours); k) use of anti-hypertensive agents; I) prior history of is cancer (excludes pre-cancerous lesions); m) life expectancy less than 1 year; n) known definite diabetes mellitus or on treatment for diabetes mellitus, autoimmune disease or genetic disease, endocrine and metabolic diseases, even on treatment, including hyperlipidemia; o) psychiatric illness; p) asthma or chronic lung disease requiring long term medications or oxygen; q) chronic infective disease, including tuberculosis, hepatitis B and C; and HIV; r) Currently participating or having participated in another clinical trial within 4 weeks prior to trial start (except for Biobank Study and SingHeart). All participants gave their written informed consent. The study protocol was approved by SingHealth CIRB and was registered at clinicaltrials.gov (NCT03531879).

Study Design:

The human clinical trial was a single center, cross-sectional study. The experimental part of the clinical trial took place on two sites: the National Heart Center of Singapore (NHCS) and the the A*Star Singapore Institute for Clinical Sciences (SICS).

Subjects were enrolled at the National Heart Center of Singapore (NHCS), where subclinical atherosclerosis and cardiovascular assessments were performed. Subclinical atherosclerosis assessments consisted of measurements of presence of plaque in the carotid, abdominal aortic or iliofemoral territory by vascular ultrasound imaging, coronary artery calcification score by computed tomography and cIMT. Cardiac structure and anatomy was also measured.

Less than a 1 week apart from the first visit, subjects went to the SICS for the food challenge.

A standardized dinner to be consumed on the evening prior to the food challenge was provided to the subjects. Standard frozen food was shipped to the subject's home. The standardized dinner consisted on a relatively low-fat meal.

A mixed-meal test (composed of 75 g glucose, 60 g palm olein and 20 g dairy protein served in a ˜337 mL liquid meal providing a total of ˜930 kcal) was served to the subjects and blood samples were drawn at different time points (total of 10 blood samples: TO/10/20/30/45/60/90/120/240/360 min) to allow analysis of several biomarkers.

Any medication/treatment initiated during the course of the trial was recorded in the eCRF.

Clinical Outcomes and Biomarkers:

Subclinical atherosclerosis assessments consisted of measurements of presence of plaque in the carotid, abdominal aortic or iliofemoral territory by vascular ultrasound imaging, coronary artery calcification score by computed tomography and cIMT. Cardiac structure and anatomy was also measured.

FIG. 4 shows the results of this study. A remarkable number of plaques was found in men and women at different sites in the body, although the subjects were healthy.

Glucose, insulin and c-peptide were quantified at 0, 10, 20, 30, 45, 60, 90, 120, 240 and 360 min. ApoB48, leptin, adiponectin, CRP, TNFα, IL-6, PAI-1, VCAM-1, ICAM-1, and E-selectin were assessed at 0, 60, 120, 240 and 360 min. Blood pressure was assessed at 0, 60, 120 and 360 min.

A physical activity tracker was used to collect Heart Rate, physical activity (calories burned, step count, distance travelled, number of floors climbed, minutes sedentary, minutes lightly active, minutes fairly active, minutes very active, activity calories) and sleep (minutes asleep, minutes awake, number of awakenings, time in bed). The fitness activity tracker was given to the subjects during the screening visit and data was collected over 5 to 7 days (before the last visit of this study).

The inventors were surprised to see that the consumption of the composition of the present invention allowed blood pressure to predict the presence or absence of atheroma plaque in an apparently healthy population (low CV risk accordingly with Framingham Score) with 91% confidence, taking into account blood pressure and Age. Best results were obtained if the blood pressure, for example the postprandial diastolic blood pressure, was measured 120 minutes after the food challenge.

Such a predictive analysis can be carried out as described hereunder.

1. Goal of Classification Analyses

Classification analyses aim to construct a model that enable to predict whether a subject belongs to one specific group or to another. For example, two class classification analyses aim to predict whether a subject has a disease or is healthy. For the scope of the present analyses, it was aimed to predict whether a subject has at least one” atherosclerosis plaque” or “no plaque”.

2. Constructing a Classification Model

Model construction and training was illustrated in FIG. 3A.:

The model was built as follows,

1. Split the Data into Two Distinct Pieces

a. A training set used by an algorithm (i.e. statistical method) to “train a model”. The training involves finding variables (i.e. measurements) and which thresholds (or coefficients) to use, in order to achieve classification. This phase learns from the data (i.e. from the class labels)

b. A Testing set to be used only for testing the performance of the trained model. This dataset shall not be used during the training phase.

3. Evaluating Model Performance

Performance evaluation is key, for both the training phase and final validation of trained models. The principles are summarized in FIG. 3B.

Briefly, once a model is trained, it will be applied on a new piece of data, not used during the training phase (e.g. a testing dataset). The model computes probabilities to be in a given class (e.g. “disease group”). A decision needs to be made based on this probability, requiring the use of a threshold. For e.g. if a subject is predicted to have the disease at 80%, one may decide he has the disease. Conversely, a subject with probability to have the disease at only 10%, may be considered healthy.

The choice of the threshold (e.g. 50%) is thus critical because it can impact the final classification for a subject whose probability is close to the threshold (e.g. probability to have the disease=49%). This will impact whether a subject is correctly classified or not.

Typically, the error is evaluated for different choice of threshold. For each given threshold, the so-called confusion matrix (see below for more details) can be computed. This matrix essentially counts the number of correctly and incorrectly classified subjects. By using different thresholds, one can generate as many confusion matrices, which in turn can be used to derived sensitivity and specificity (at different thresholds). These two metrics are often presented in the form or Receiving Operating Curve (ROC); which summarizes in one single plot the model performance over several choices of thresholds.

Required Parameters:

age of the subject (in years)

either the diastolic blood pressure (mm Hg), 2 hours after meal

or the systolic blood pressure (mm Hg), 2 hours after meal

then,

for the DIABP model, the computations would be:

age_standardized=(age −47.92105)/4.200835

DIABP_standardized=(DIABP −71.44079)/10.017973

logit_score=0.5108+(−0.3682*DIABP_standardized)+(−0.3998*age_standardized)

odds_score=exp(logit_score)

probability=odds_score/(1+odds_score)

the SYSBP model, these computations are:

age_standardized=(age −47.92105)/4.200835

SYSBP_standardized=(SYSBP −114.41447)/13.522423

logit_score=0.5253+(−0.5403*SYSBP_standardized)+(−0.3998*age_standardized)

odds_score=exp(logit_score)

probability=odds_score/(1+odds_score)

As a result,

a man, 48 years old with 140 mm Hg postprandial systolic blood pressure 2 h after consumption of the composition of the present invention has a risk of having atheroma plaque of 62%.

a women, 50 years old with 82 mm Hg postprandial diastolic blood pressure 2 h after consumption of the composition of the present invention has a risk of having atheroma plaque of 53%.

Using this model, the presence or absence of atheroma plaque in an apparently healthy population (low CV risk accordingly with Framingham Score) was predicted with 91% confidence taking into account postprandial diastolic blood pressure at 120 min and age of the subject.

1. (canceled)

2. Composition for use in accordance with claim 4 wherein the diagnostic method is a diagnostic method to predict the risk of atheroma plaque formation in a subject.

3. Composition for use in accordance with claim 2 wherein the risk of atheroma plaque formation is used as an indicator to predict the likelihood of the presence or development of cardiovascular disorders.

4. A diagnostic method comprising the steps of
determining the age of the subject
administering to the subject a nutritional composition comprising 15−70 g fat, 60−90 g carbohydrates and 15−35 g protein,
measuring the blood pressure of that subject after administration of the composition, and
wherein an elevated blood pressure compared to a reference value specific for the age group of the subject indicates an increased risk for atheroma plaque formation.

5. Method in accordance with claim 4, wherein the diagnostic method further includes that the measured blood pressure and the age of the subject are subjected to an algorithm that produces an indicative figure and that indicative figure is compared to corresponding indicative reference figures for the age group of the subject.

6. Method in accordance with claim 4, wherein the diagnostic method further includes that the measured blood pressure and the age of the subject are mean-centered and unit-scaled.

7. Method in accordance with one of claim 4, wherein the diagnostic method further includes that the postprandial systolic blood pressure of the subject is measured and an increased risk of atheroma plaque formation is predicted, if the value for the mean-centered and unit-scaled systolic blood pressure is bigger than −0.002× the mean-centered and unit-scaled age of the subject +0.57.

8. Method in accordance with claim 7, wherein the diagnostic method further includes that an increased risk of atheroma plaque formation is predicted if (systolic blood pressure −114.4)/13.5 >−0.002× (age of the subject −47.9)14.2)+0.57.

9. Method in accordance with claim 4, wherein the diagnostic method further includes that the postprandial diastolic blood pressure of the subject is measured and an increased risk of atheroma plaque formation is predicted, if the value for the mean-centered and unit-scaled diastolic blood pressure is bigger than 0.001× the mean-centered and unit-scaled age of the subject +0.43.

10. Method in accordance with claim 9, wherein the diagnostic method further includes that an increased risk of atheroma plaque formation is predicted if (diastolic blood pressure −71.4)/10 >0.001× (age of the subject −47.9)/4.2)+0.43.

11. Method in accordance with claim 4, wherein the diagnostic method further includes that the blood pressure is measured 65−360 min after the nutritional composition was administered.

12. Method in accordance with claim 4, wherein the nutritional composition contains 500−1000 kcal per serving.

13. Method in accordance with claim 4, wherein the nutritional composition comprises 20−71 g fat, 70−80 g carbohydrates and 20−30 g protein.

14. Method in accordance with claim 4, wherein the composition further comprises vitamins and minerals.

15. Method in accordance with claim 4, wherein the composition is a drinkable composition with a volume in the range of 200 ml −400 ml.

16. Method in accordance with claim 4, wherein the diagnostic method is implemented through a computer/digital system.
